(19) United States
(12) Patent Application Publication
(10) Pub. No.: US 2006/0018951 A1
(43) Pub. Date: Jan. 26, 2006

(54) pH-responsive film for intravaginal delivery of a beneficial agent

(75) Inventors: Manoj Maniar, Fremont, CA (US); Shoreh Parandoosh, Palo alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SRI International, Menlo Park, CA (US)

(21) Appl. No.: 10/912,814

(22) Filed: Aug. 6, 2004

Related U.S. Application Data
(60) Provisional application No. 60/560,739, filed on Aug. 8, 2003.

(51) Int. Cl. A61K 9/14; A61K 9/70

(52) U.S. Cl. 424/443; 424/487

(57) ABSTRACT

The present invention provides a delivery system for the intravaginal administration of prophylactic and therapeutic agents. In one embodiment, the invention provides a pH-responsive, biocompatible film for intravaginal administration of a beneficial agent, comprising a biocompatible, hydrophilic polymer that is positively charged at a first pH and in electronically neutral form at a higher pH; an effective amount of a beneficial agent; and, optionally, at least one film-forming binder. The pH responsive film may also include other additives such as plasticizers, sustained release polymers, antioxidants, and antimicrobial agents. In another embodiment, the pH-responsive film of the present invention comprises a laminated composite of (a) a bioadhesive layer that serves to affix the film to a mucosal surface within the vagina and, laminated thereto, (b) at least one reservoir layer comprising at least one beneficial agent and a biocompatible hydrophilic polymer. The pH responsive films of the present invention can be used for contraception, treatment and/or prevention of viral infections, treatment of vaginal infections, relief of vaginal itch, vaginal cleansing, and enhancement of vaginal lubrication.

### CROSS-REFERENCE TO RELATED APPLICATIONS

This application claims priority to U.S. Application Ser. No. 60/560,739, filed Aug. 8, 2003, the disclosure of which is incorporated herein by reference.

### TECHNICAL FIELD

This present invention relates generally to pH-responsive films, preferably those with an interpenetrating network. The films are useful in methods and delivery systems for the administration of beneficial agents, and more particularly relates to a delivery system for the intravaginal administration of prophylactic and therapeutic agents. The methods and delivery systems of the invention have utility in a variety of technical fields, including drug delivery and contraception.

### BACKGROUND OF THE INVENTION

The vaginal environment is influenced by several biologic factors, including changes caused by local or systemic disorders and diseases, changes associated with menopause or menstrual cycles, pharmacotherapeutic treatment of such conditions, and other health practices such as sexual and hygiene measures. The normal, acidic pH of a human vagina is 3.8 to 4.5. An increase in vaginal pH can result from non-infectious causes, such as the presence of semen in the vagina after intercourse, as well as from various types of infections, including trichomoniasis vaginitis, bacterial vaginosis, streptococcal bacterial vaginitis, and desquamative inflammatory vaginitis.

Vaginal pH affects the viability of many organisms. For instance, human immunodeficiency virus (HIV) appears to survive best in a neutral pH rather than in an acidic pH. Further, pH levels below 5.5 inactivate several harmful bacteria including those causing gonorrhea and bacterial vaginosis. The optimum pH value for sperm migration and survival in the cervical mucus is between 7.0 and 8.5. Below pH levels of 6.9 sperm die at a rate that increases with lowering pH.

While physical barrier methods of contraception, particularly condoms, generally prevent pregnancy as well as the transmission of most sexually transmitted diseases (STDs), many couples still do not use such methods. Additionally, there are several disadvantages associated with spermicidal creams and gels. For instance, creams and gels tend to melt quickly and thus, are inconvenient, messy to use, and easily discharged from the vagina, thereby limiting their effectiveness and requiring repeated dosing.

To help prevent unwanted pregnancies and/or prevent the spread of HIV and other STDs, researchers have been trying to develop effective products that people will use more consistently. For instance, a new product designed to create a physical barrier, thereby keeping pathogens away from human cells and preventing conception, is the Invisible Condom® (developed at Laval University in Quebec, Canada). The Invisible Condom is a polymer-based gel that hardens upon increased temperature after insertion into the vagina or rectum. In the laboratory, it has been shown to effectively block the transmission of HIV and herpes simplex virus. The barrier breaks down and liquefies after several hours.

Another approach has been in the development of lubricants and gels that contain microbicides, which are to be applied during sexual activity. The first compound to be tested in Phase III clinical trials as a microbicidal candidate was nonoxynol-9, which was approved by the FDA as a spermicidal contraceptive and has been on the market for many years in gel form. It has been reported; however, that nonoxynol-9 promotes, rather than prevents, HIV transmission because it irritates the cells lining the vagina, providing viruses with an entry point through the damaged tissue. Federal Registrar, Vol. 68, No. 11 (Jan. 16, 2003).

Another proposed microbicide that is ready to enter Phase III clinical trials is a carbopol polymer gel (BufferGel®, manufactured by ReProtect, LLC, Baltimore, Md.) that is osmotically balanced with physiological salts. A commercial product that combines both barrier and chemical forms of contraception is VCF® (manufactured by Apothecus, Inc., Great Neck, N.Y.), a vaginal contraceptive film, which dissolves into a gel and blocks the cervix. VCF is reported to be effective for up to three hours, but does not adequately protect users from HIV and other STDs.

Accordingly, there remains a need in the art for an effective product that will prevent unwanted pregnancies and/or prevent the transmission of STDs including HIV, and that does not suffer from the disadvantages of currently available products. An ideal product would be easy to use and not readily discharged. To encourage the use of such a product, it should be designed to be inexpensive, convenient, unobtrusive, and non-irritating to both partners.

### BRIEF SUMMARY OF THE INVENTION

In one aspect, the present invention provides a pH-responsive film comprising:
(a) a biocompatible, hydrophilic polymer that is positively charged at a first pH and in electronically neutral form at a higher pH; and
(b) an alkylene oxide polymer or copolymer.

Without intending to be bound by theory, it is believed that advantages in the present invention are obtained by the development of an interpenetrating network in the pH-responsive film, that is formed between the hydrophilic polymer component and the alkylene oxide polymer or copolymer component. The interpenetrating network facilitates the incorporation of biological agents, pH-adjusting agents and the like, which in combination with the film can help to reduce the transmission of, for example, viral infections.

Accordingly, in another aspect, the present invention provides a pH-responsive film for administration of a beneficial agent, comprising an effective amount of a beneficial agent and a biocompatible, hydrophilic polymer that is positively charged at a first pH and in electronically neutral form at a higher pH and an alkylene oxide polymer or copolymer. In some embodiments, the film will include at least one additional film component, such as for example, a cellulose ether. The films provided herein are capable of delivering a wide variety of beneficial agents, alone or in combination with a lubricant.

In another aspect, the present invention provides a laminated composite film having a bioadhesive layer that serves to affix the film to a mucosal surface within the vagina. A reservoir layer comprising the beneficial agent and a biocompatible hydrophilic polymer which is laminated to the bioadhesive layer.

In a related group of embodiments, the invention provides a laminated composition having a reservoir layer that comprises a first region containing one beneficial agent and a second region containing a second beneficial agent.

In still another aspect, the invention provides a method of treating or preventing pH-responsive disorders in a female individual, by positioning in the vaginal passage of the individual a pH-responsive film for the administration of a beneficial agent, wherein the pH-responsive responsive film includes: an effective amount of an ionizable beneficial agent and a biocompatible, hydrophilic polymer that is positively charged at a first pH and in electronically neutral form at a higher pH. When the pH is equal to or above 7, the film releases the beneficial agent into the vagina. Drug release may or may not be gradual. That is, sustained release is preferred for some uses, while immediate, complete release is desirable for other uses.

In yet another aspect, the present invention provides a method of contraception in a female individual, by positioning in the vaginal passage of the individual prior to sexual intercourse a pH-responsive film for the administration of a beneficial agent, wherein the pH-responsive film comprises an effective amount of an ionizable beneficial agent selected to effect contraception (e.g., a spermicide); and, a biocompatible, hydrophilic polymer that is positively charged at a first pH and in electronically neutral form at a higher pH. When the pH is greater than or equal to 7, the agent is released as described above.

These and other objects and features of the present invention will become more fully apparent from the following description and appended claims, or may be learned by the practice of the invention as set forth hereinafter.

### BRIEF DESCRIPTION OF THE DRAWINGS

Not applicable.

### DETAILED DESCRIPTION OF THE INVENTION

I. Definitions and Nomenclature

As used in this specification and the appended claims, the singular forms “a,” “an” and “the” include plural referents unless the context clearly dictates otherwise. Thus, for example, reference to “an active agent” includes a single active agent as well a two or more different active agents in combination, reference to “a pharmaceutically acceptable carrier” includes mixtures of two or more such carriers as well as a single carrier, and the like.

In describing and claiming the present invention, the following terminology will be used in accordance with the definitions set out below.

The terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition (e.g., pregnancy). The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. “Treating” a patient by administering a beneficial agent includes prevention of a particular disorder or unwanted physiological event as well as treatment of a clinically symptomatic individual by inhibiting or causing regression of a disorder or disease.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the individual, the particular beneficial agent or agents, and the like. Thus, it is not always possible to specify an exact “effective amount.” However, an appropriate “effective” amount in any individual case may be determined by one of ordinary skill in the art using routine experimentation.

The term “controlled release” refers to a formulation, dosage form, or region thereof from which release of a beneficial agent is not immediate, i.e., with a “controlled release” dosage form, administration does not result in immediate release of the beneficial agent in an

absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

The term “sustained release” (synonymous with “extended release”) is used in its conventional sense to refer to a formulation, dosage form, or region thereof that provides for gradual release of a beneficial agent over an extended period of time, and that preferably, although not necessarily, results in substantially constant blood levels of the agent over an extended time period.

The term “biocompatible” refers to a material that is not biologically undesirable, i.e., the material may be incorporated into a formulation administered to a patient generally without resulting in substantial undesirable biological effects.

The term “pharmaceutically acceptable,” as used to refer to a pharmaceutical carrier or excipient, is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree.

The term “ionizable” refers to a compound containing at least one functional group that (a) bears a positive or negative charge (i.e., is “ionized”) and is therefore associated with a counterion of opposite charge, or (b) is electronically neutral but ionized at a higher or lower pH. Thus, ionizable compounds include quaternary ammonium salts as well as uncharged amines, and carboxylate moieties as well as uncharged carboxyl groups.

The term “naturally occurring” refers to a compound or composition that occurs in nature, regardless of whether the compound or composition has been isolated from a natural source or chemically synthesized.

The term “polymer” as used herein refers to a molecule containing a plurality of covalently attached monomer units, and includes branched, dendrimeric and star polymers as well as linear polymers. The term also includes both homopolymers and copolymers, e.g., random copolymers, block copolymers and graft copolymers, as well as uncrosslinked polymers and slightly to moderately to substantially crosslinked polymers.

II. The pH-Responsive Film

The present invention provides a pH-responsive, biocompatible film comprising a biocompatible, hydrophilic polymer that is positively charged at a first pH and in electronically neutral form at a higher pH; and, an alkylene oxide polymer or copolymer component. In related aspects, discussed below, the films will contain a beneficial agent for intravaginal administration.

A. The Biocompatible, Hydrophilic Polymer

The biocompatible, hydrophilic polymer is preferably a naturally occurring, water swellable polymer. The term “hydrophilic” is used in its conventional sense to indicate that the polymer is compatible with aqueous fluids such as those present in the human body, e.g., within the vagina. More specifically, the hydrophilicity of the polymer may be defined in terms of a partition coefficient P, which is the ratio of the equilibrium concentration of a compound in an organic phase to that in an aqueous phase. The present hydrophilic polymer has a log P value less than 1.0, typically less than about 0.5, where P is the partition coefficient of the polymer between octanol and water.

Preferred polymers are bioadhesive, i.e., exhibit a tendency to adhere to the surface of mucosal tissue, thereby facilitating adhesion of the film to the vaginal walls. The polymer should also be swellable, such that upon absorption of an aqueous fluid, the film swells, enabling release of a beneficial agent (see below) from the interior of the film through the swollen polymer matrix. In addition, the polymer should be bioerodible, meaning that it slowly dissolves, gradually hydrolyzes, and/or physically erodes within an aqueous medium.

Particularly preferred biocompatible, hydrophilic polymers are positively charged at a first pH and in electronically neutral form at a higher pH. Such polymers are advantageous in films used for intravaginal delivery of an ionizable drug or other beneficial agent, insofar as the charged polymer will ionically bind the agent at low pH, but at a higher pH the ionic interaction will cease and the agent will be gradually released from the uncharged film. As noted above, the healthy vagina has a pH of about 4, while many vaginal disorders increase the pH to above 7. Additionally, the presence of semen also increases the pH to above 7. Therefore, pH-responsive polymers that are ionized at low pH but electronically neutral at a basic pH are optimal for delivering spermicides or agents to treat many vaginal disorders. Ideally, the hydrophilic polymer has a pKa of≦6, so that the film will deliver the beneficial agent upon exposure to a pH of about 7 or higher.

The amount of hydrophilic polymer used in the present invention is typically, although not necessarily, in the range of about 5 to about 90 wt. %, preferably in the range of about 7.5 to about 65 wt. %, more preferably about 20 to about 45 wt. %.

Preferably, the biocompatible, hydrophilic polymers are polysaccharides and cellulosic polymers, such as polysaccharides and cellulosic polymers bearing free or protected (e.g. acetylated) amino groups. Particularly preferred biocompatible, hydrophilic polymers are chitosan and glycosaminoglycans.

Chitosan, as is well known, is partially or wholly deacetylated chitin, which is a cellulose-like polymer consisting predominantly of unbranched chains of β-(1→4)-2-acetamido-2-deoxy-D-glucose (also termed “N-acetyl-D-glucosamine”) residues that is found in fungi, yeasts, marine invertebrates and arthropods, where it is a principal component in the exoskeletons. It will be appreciated that chitosan in the form of wholly deacetylated chitin has a higher water solubility and ionically binds ionizable agents more strongly than partially deacetylated chitin. The chitosan herein may be partially or wholly deacetylated chitin, depending upon the desired properties of the film (i.e., degree and rate of dissolution, ionic binding strength, etc.). While the partially deacetylated chitin shown below illustrates contiguous portions of acetylated and deacetylated chitin, one of skill in the art will apprecitate that partially deacetylated chitin also includes those forms wherein portions of deacetylated chitin are interrupted by the acetylated portions.

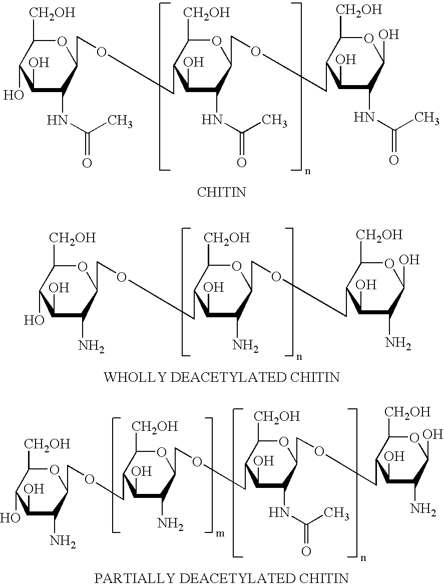

At a pH of 4, chitosan is fully protonated. Chitosan has a pKa of about 8, which means that at elevated pH levels, the polymer will be electronically neutral. Chitosan also exhibits bioadhesion, thus facilitating transmucosal absorption by adhering to mucosal surfaces of the vaginal walls. It should also be noted that chitosan is a water-swellable polymer, and can therefore release beneficial agent from the interior of the film through the swollen matrix. The chitosan used in accordance with the present invention generally has a weight average molecular weight in the range of about 15,000 to about 1,000,000, preferably in the range of about 30,000 to about 300,000.

Chitosan may be derivatized in various ways. For instance, some of the known reagents used to make such derivatives of chitosan, include for example, ethylene and propylene oxide, carboxylic acids, quaternary ammonium reagents, monochloroacetic acid and various anhydrides. A typical salt, for example, might include chitosan lactate, chitosan epoxysuccinate, chitosan monochloroacetate, chitosan salicylate, chitosan itaconate, chitosan pyrrolidone carboxylate, chitosan glycolate, chitosan hydrochloride, chitosan ascorbate, chitosan acetate, chitosan citrate, chitosan benzoate, chitosan nicotinate, chitosan malate, chitosan aspartate, chitosan glutamate, chitosan succinate, chitosan formate, chitosan pyruvate, chitosan propionate, chitosan tartrate and mixtures thereof.

Glycosaminoglycans are well known, naturally occurring polysaccharides containing disaccharide repeating units of hexosamine and hexose or hexuronic acid and may contain sulfate groups. Representative glycosaminoglycans include, but are not limited to: hyaluronan, hyaluronic acid or derivatives thereof such as hylan; heparin; heparan; chondroitin; keratan; and sulfates of such materials.

C. Alkylene Oxide Polymer or Copolymer

Another component of the present films include alkylene oxide polymers or copolymers. These film components are selected to allow processing of the combination (e.g., chitosan and alkylene oxide copolymer) into a film of a desired thickness and flexibility. Without intending to be bound by theory, it is also believed that selection of suitable polymer or copolymer components allows an interpenetrating network to develop in the film, due to hydrogen bonding between the biocompatible, hydrophilic polymer and the alkylene oxide polymer or copolymer component. Preferred alkylene oxide polymer or copolymer components are also biocompatible and thus suitable for internal use. Additionally, the alkylene oxide polymer or copolymer component is also melt-extrudable and gradually water-soluble. Still further preferred are those alkylene oxide polymer or copolymer components that are bioerodible. The total amount of this component in the film is in the range of about 2 to about 85 wt. %, preferably about 3 to about 35 wt. %. In some embodiments, the polymer or copolymer component will be combined with still another film component, for example, a cellulose ether component, as discuss below.

Exemplary alkylene oxide polymer or copolymers are hydrophilic polymers, including, without limitation, poly(alkylene oxides) such as polyethylene oxide (PEO) and poloxamers (i.e., copolymers of ethylene oxide and propylene oxide such as Pluronic® as manufactured by BASF), with poloxamers representing preferred components.

In other embodiments, the alkylene oxide polymer or copolymer can be replaced with a polyvinyl alcohol, polylactide, poly(lactide-co-glycolide), polysorbate, poly(oxyethylated)glycerol, poly(oxyethylated)sorbitol, poly(oxyethylated)glucose, cellulosic polymers, and mixtures thereof.

In a particularly preferred embodiment, a poloxamer is used in combination with a cellulose ether, e.g., hydroxyethyl cellulose, hydroxypropyl cellulose, or hydroxypropyl methylcellulose (HPMC), with HPMC most preferred. The combination of a poloxamer and HPMC has been found to be particularly advantageous. PEO homopolymers (non-ionic surfactants having good lubricity) are useful film-forming polymers for purposes of the present invention.

C. Additional Film Components

Plasticizers may be added to the pH-responsive film to enhance softness and manufacturability. Examples of suitable plasticizers include glycerin, glycerides such as triglyceride, sorbitol, propylene glycol, polyethylene glycol, triacetin, triethyl citrate (TEC), acetyl triethyl citrate (ATEC) and other citrate esters, and glycerides, particularly monoglycerides. The amount of the plasticizer exerts an influence on crystallinity, flexibility, heat resistance and the like of the film. When the amount is too high, the crystallinity and heat resistance lower. When the amount is too low, sufficient flexibility is not obtained. From such a standpoint, it is preferable that the total amount of the plasticizer in the film is from about 1 to about 60 wt. %. Preferably still, the amount of the plasticizer is from about 5 to about 50 wt. %.

Additional sustained release polymers may be added for increasing the agent release period. Examples include very high molecular weight polyethylene oxide.

Other optional additives include antioxidants, i.e., agents inhibit oxidation and thus prevent the deterioration of preparations by oxidation. Suitable antioxidants include, by way of example and without limitation, ascorbic acid, ascorbyl palmitate, butylated hydroxyanisole, butylated hydroxytoluene, hypophophorous acid, monothioglycerol, sodium ascorbate, sodium formaldehyde sulfoxylate and sodium metabisulfite and others known to those of ordinary skill in the art. Other suitable antioxidants include, for example, vitamin C, butylated hydroxytoluene (BHT), butylated hydroxyanisole (BHA), sodium bisulfite, vitamin E and its derivatives, propyl gallate, sulfite derivatives, and others known to those of ordinary skill in the art.

Antimicrobial agents may also be added to the pH-responsive film. Antimicrobial agents function by destroying microbes, preventing their pathogenic action, and/or inhibiting their growth. Desirable properties of antimicrobial agents include, but are not limited to: (1) the ability to inactivate bacteria, viruses and fungi, (2) the ability to be effective within minutes of application and long after initial application, (3) cost, (4) compatibility with other components of composition, (5) stability at ambient temperature, and (6) lack of toxicity.

III. pH-Responsive Film for the Administration of a Beneficial Agent

As noted above, in a related aspect, the present invention provides a pH-responsive film for the administration of a beneficial agent. In general, these films utilize the components that have been described above, but also contain an effective amount of a beneficial agent.

B. Beneficial Agents

The beneficial agent may be any prophylactic agent or therapeutic agent suitable for vaginal administration. Preferably, the beneficial agent achieves a local rather than a systemic effect, meaning that the agent functions in the desired beneficial manner without entering the bloodstream. Therefore, “local” effects include spermicidal activity, treatment of a vaginal condition or disorder, prevention or treatment of a sexually transmitted disease, and the like. Suitable beneficial agents that may be administered using the present pH-responsive film thus include, without limitation, spermicidal agents, antiviral agents, anti-inflammatory agents, local anesthetic agents, anti-infective agents, that latter including antibiotics, antifungal agents, antiparasitic agents, acids, lubricants and mixtures thereof. Exemplary agents are as follows:

Spermicidal agents include nonylphenoxypolyethoxy ethanol (sold under the tradename “Nonoxynol-9“), p-diisobutylphenoxy polyethanol (“Octoxynol-9“), benzalkonium chloride, p-methanyl phenylpolyoxyethylene ether (Menfegol), chlorhexidine, polyoxyethylene oxypropylene stearate, ricinoleic acid, glycerol ricinoleate, methyl benzethonium chloride, and mixtures thereof. Nonoxynol-9, Octoxynol-9, benzalkonium chloride, and Menfegol being preferred.

Antiviral agents include nucleoside phosphonates and other nucleoside analogs, AICAR (5-amino-4-imidazolecarboxamide ribonucleotide) analogs, glycolytic pathway inhibitors, anionic polymers, and the like, more specifically: antiherpes agents such as acyclovir, famciclovir, foscamet, ganciclovir, idoxuridine, sorivudine, trifluridine, valacyclovir, and vidarabine; and other antiviral agents such as abacavir, adefovir, amantadine, amprenavir, cidofovir, delviridine, 2-deoxyglucose, dextran sulfate, didanosine, efavirenz, indinavir, interferon alpha, lamivudine, nelfinavir, nevirapine, ribavirin, rimantadine, ritonavir, saquinavir, squalamine, stavudine, tipranavir, valganciclovir, zalcitabine, zidovudine, zintevir, and mixtures thereof. Still other antiviral agents are glycerides, particularly monoglycerides, that have antiviral activity. One such agent is monolaurin, the monoglyceride of lauric acid.

Anti-inflammatory agents include corticosteroids, e.g., a lower potency corticosteroid such as hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, or methylprednisolone, or a higher potency corticosteroid such as clobetasol propionate, betamethasone benzoate, betamethasone diproprionate, diflorasone diacetate, fluocinonide, mometasone furoate, triamcinolone acetonide, and mixtures thereof.

Local anesthetic agents include acetamidoeugenol, alfadolone acetate, alfaxalone, amucaine, amolanone, amylocaine, benoxinate, benzocaine, betoxycaine, biphenamine, bupivacaine, burethamine, butacaine, butaben, butanilicaine, buthalital, butoxycaine, carticaine, 2-chloroprocaine, cocaethylene, cocaine, cyclomethycaine, dibucaine, dimethisoquin, dimethocaine, diperadon, dyclonine, ecgonidine, ecgonine, ethyl aminobenzoate, ethyl chloride, etidocaine, etoxadrol, β-eucaine, euprocin, fenalcomine, fomocaine, hexobarbital, hexylcaine, hydroxydione, hydroxyprocaine, hydroxytetracaine, isobutyl p-aminobenzoate, ketamine, leucinocaine mesylate, levobupivacaine, levoxadrol, lidocaine, mepivacaine, meprylcaine, metabutoxycaine, methohexital, methyl chloride, midazolam, myrtecaine, naepaine, octacaine, orthocaine, oxethazaine, parethoxycaine, phenacaine, phencyclidine, phenol, piperocaine, piridocaine, polidocanol, pramoxine, prilocaine, procaine, propanidid, propanocaine, proparacaine, propipocaine, propofol, propoxycaine, pseudococaine, pyrrocaine, risocaine, salicyl alcohol, tetracaine, thialbarbital, thimylal, thiobutabarbital, thiopental, tolycaine, trimecaine, zolamine, phenol, and mixtures thereof.

Antibiotic agents include those of the lincomycin family, such as lincomycin per se, clindamycin, and the 7-deoxy, 7-chloro derivative of lincomycin (i.e., 7-chloro-6,7,8-trideoxy-6-[[(1-methyl-4-p-ropyl-2-pyrrolidinyl)carbonyl]amino]-1-thio-L-threo-α-D-galacto-octopyranoside); other macrolide, aminoglycoside, and glycopeptide antibiotics such as erythromycin, clarithromycin, azithromycin, streptomycin, gentamicin, tobramycin, amikacin, neomycin, vancomycin, and teicoplanin; antibiotics of the tetracycline family, including tetracycline per se, chlortetracycline, oxytetracycline, tetracycline, demeclocycline, rolitetracycline, methacycline and doxycycline; and sulfur-based antibiotics, such as the sulfonamides sulfacetamide, sulfabenzamide, sulfadiazine, sulfadoxine, sulfamerazine, sulfamethazine, sulfamethizole, and sulfamethoxazole; streptogramin antibiotics such as quinupristin and dalfopristin; and quinolone antibiotics such as ciprofloxacin, nalidixic acid, ofloxacin, and mixtures thereof.

Antifungal agents include miconazole, terconazole, isoconazole, itraconazole, fenticonazole, fluconazole, ketoconazole, clotrimazole, butoconazole, econazole, metronidazole, clindamycin, 5-fluorouracil, amphotericin B, and mixtures thereof.

Other anti-infective agents include miscellaneous antibacterial agents such as chloramphenicol, spectinomycin, polymyxin B (colistin), and bacitracin, anti-mycobacterials such as such as isoniazid, rifampin, rifabutin, ethambutol, pyrazinamide, ethionamide, aminosalicylic acid, and cycloserine, and antihelminthic agents such as albendazole, oxfendazole, thiabendazole, and mixtures thereof.

The beneficial agent may also be an acid, having a pKa of≧3. Preferably, the pKa of the acid is about 3. Suitable acids generally although not necessarily contain at least two acidic groups, e.g., carboxylic, sulfonic, and/or phosphonic acid groups. It is also preferred that the acid is an organic acid. Preferably, the organic acid is monomeric and has the structural formula [R(LₓCOOH)_{y}]_{z} wherein: R is selected from C₁-C₁₂ alkyl, C₂-C₁₂ alkenyl, C₅-C₁₆ aryl, and C₅-C₁₆ heteroaryl (including substituted such moieties); L is C₁-C₈ alkylene or C₂-C₈ alkenylene; x is 0 or 1; y is an integer in the range of 2 to 8 inclusive; and z is 1, 2 or 3, with the proviso that if z is 2 or 3, the distinct R groups are covalently linked to each other. More preferably, the organic acid is selected from lactic, citric, and hexanoic acids. Generally, y is 2 to 4 and z is 1. Lactic acid is most preferred. In one embodiment, the biocompatible, hydrophilic polymer comprises a salt formed with the organic acid. In this case, the composition further comprises excess organic acid.

The beneficial agent may also be a lubricant. Suitable lubricants include, but are not limited to, slippery solids such as talc, magnesium stearate, calcium stearate, stearic acid, hydrogenated vegetable oils (e.g., peanut oil, cottonseed oil, sunflower oil, sesame oil, olive oil, corn oil, and soybean oil), mineral oil, glycerin, sorbitol, mannitol, polyethylene glycol, other glycols, sodium lauryl sulfate, zinc stearate, ethyl oleate, ethyl laureate, agar, and mixtures thereof. Other embodiments of the invention include a pH-responsive film having at least two beneficial agents, one of which is a lubricant. Preferably, the two beneficial agents are a lubricant and an acid.

Any of the beneficial agents may be administered in the form of a salt, ester, amide, prodrug, conjugate, active metabolite, isomer, fragment, analog, or the like, provided that the salt, ester, amide, prodrug, conjugate, active metabolite, isomer, fragment, or analog is pharmaceutically acceptable and pharmacologically active in the present context. Salts, esters, amides, prodrugs, conjugates, active metabolites, isomers, fragments, and analogs of the agents may be prepared using standard procedures known to those skilled in the art of synthetic organic chemistry and described, for example, by J. March, Advanced Organic Chemistry: Reactions, Mechanisms and Structure, 5th Edition (New York: Wiley-Interscience, 2001).

For example, acid addition salts are prepared from a drug in the form of a free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

The amount of the beneficial agent(s) in the film will typically range from about 5 to about 50 wt. % based on the total weight of the film, preferably from about 5 to about 35 wt. %.

The beneficial agent is blended homogeneously with the biodegradable polymer so that the agent is evenly distributed through the film. Upon contact with vaginal fluid, the film gradually degrades, releasing the beneficial agent in the proper dosage and at the proper rate to perform its function. The beneficial agent is selected for its dissolution profile and compatibility with the biocompatible polymer.

In certain preferred embodiments, the pH-responsive films of the present invention comprise of from 20 to 60 weight percent chitosan lactate, of from 3 to 35 weight percent of a poloxamer copolymer, of from 5 to 45 weight percent of hydroxypropyl methylcellulose, and of from 5 to 45 weight percent glycerin.

IV. Composite Films

In yet another aspect, the present invention provides a composite film which is a pH-responsive, laminated composite comprising:
(a) a bioadhesive layer that serves to affix the film to a mucosal surface within the vagina and, laminated thereto,
(b) a reservoir layer comprising a beneficial agent and a biocompatible hydrophilic polymer.

In some embodiments, the reservoir layer comprises a first layer having the beneficial agent and a second layer having the biocompatible hydrophilic polymer.

The pH-responsive film can be manufactured for controlled release in a high pH environment, so that the beneficial agent can be released gradually over an extended time period, e.g., for delivery of an antiviral agent. For instance, controlled release can be achieved by wherein the first layer of the reservoir layer further comprises a controlled release polymer and the second layer of the reservoir layer is, or further comprises, a pH-responsive material. For those embodiments in which the biocompatible hydrophilic polymer also functions as a pH-responsive material, the controlled release polymer can be selected from carbomers, poly(alkylene oxides), and cellulose ethers. Carbomers include any polymers in the family, e.g., carboxypolyalkylenes, which may be obtained commercially under the Carbopol® trademark. Preferably, the poly(alkylene oxide) is poly(ethylene oxide) and the cellulose ether is hydroxypropyl methylcellulose.

The biocompatible hydrophilic polymer can also be selected to function as the controlled release polymer. In this case, a pH-responsive material is typically selected from: the chitosans provided above (e.g., chitosan lactate), cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropyl methylcellulose phthalate, hydroxypropyl methylcellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the tradename “Eudragit®”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac).

In some embodiments, the pH-responsive film is manufactured to provide immediate release of the beneficial agent upon an increase in pH beyond 7, e.g., for delivery of a spermicide.

In other embodiments, the reservoir layer of the pH-responsive film comprises two or more beneficial agents. When two or more beneficial agents are used, the reservoir layer can have two or more distinct regions, each containing a different beneficial agent (for example, if the beneficial agents are incompatible). The regions may also be layers of a bilayer. A pH-responsive composite film having two distinct regions, each containing a different beneficial agent, can also be constructed to provide two different release profiles (e.g., a layer that immediately releases, for example, a spermicide upon an increase in pH to greater than 7, and a layer that provides gradual release of, for example, an antiviral agent).

The beneficial agent can be released from the pH-responsive composite film of the present invention by a variety of mechanisms. For instance, the beneficial agent may be released osmotically. One type of osmotic release occurs when the beneficial agent dissolves and/or degrades upon swelling of the biocompatible, hydrophilic polymer and is thereby released into the vagina. Another type of osmotic release occurs when the beneficial agent, e.g. a non-ionizable agent, is displaced, whereby the biocompatible hydrophilic polymer imbibes an aqueous or biological fluid and swells to push the beneficial agent to the surface of the film, thereby releasing the beneficial agent into the vaginal passage.

When the beneficial agent is an ionizable agent, release from the pH-responsive composite film occurs in part as a result of dissolution and/or degradation, and in part as a result of neutralization of the ionizable polymer at elevated pH, such that the polymer no longer ionically binds the agent.

V. Film Structure and Manufacture

In general, the films of the present invention are manufactured using methods standard in the art, e.g., solvent-evaporation film casting in which all components of the pH-responsive film are mixed together, cast onto a substrate using a casting knife, shaped to the desired dimensions, and dried. In particular, the biocompatible hydrophilic polymer and, when present, the additional components, are mixed with a suitable solvent, such as water. The beneficial agent and, when present, the plasticizer, are added to the solution. Suitable solvents for manufacturing the films include inert inorganic and organic solvents that do not adversely harm the materials and the final laminated wall.

Preferably, the thickness of the wet film is in the range of about 3 to about 6 mil, more preferably about 4 mil. The wet film may be air dried for a period of time, such as 10-12 hours, and then vacuum dried at a temperature in the range of about 20 to 90° C. for, generally, about 1-5 hours. Other layers may be then laminated to this initial structure.

Other standard manufacturing procedures suitable for use herein are described in Modern Plastics Encyclopedia 46: 62-70 (1969), Riegel's Handbook of Industrial Chemistry, 9th Edition, J. Kent, Ed. (New York: Chapman & Hall, 1992), Handbook of plastic Materials and Technology, I. Rubin, Ed. (New York: John Wiley & Sons, 1990), and in Remington, supra.

VI. Methods of Use

The pH-responsive films, films with beneficial agents and composite films of the present invention may be used for the following applications:
a) contraception, either with or without an additional agent such as Nonoxynol-9;
b) treatment/prevention of viral infections, such as genital herpes, human papilloma virus, and HIV, by release of an antiviral agent (e.g., acyclovir for genital herpes);
c) treatment of vaginal infections, such as vaginitis, vaginal candidiasis, including genital candidiasis caused by Candida albicans, trichomoniasis, bacterial vaginosis, chlamydial infections, and gonorrhea, by release of a suitable agent (e.g., tetracycline for gonorrhea; metronidazole for trichomoniasis);
d) relief of vaginal itch caused by non-specific yeast infections by administering an appropriate medication, such as an anti-inflammatory agent or local anesthetic agent;
e) vaginal cleansing, by coating the vaginal wall during insertion of the film with suitable agents; and
f) enhancement of vaginal lubrication.

The present invention provides for a method of treating or preventing pH-responsive diseases in a female individual, comprising: positioning in the vaginal passage of the female individual a pH-responsive film for the administration of a beneficial agent, wherein the pH-responsive film comprises an effective amount of an ionizable beneficial agent; and a biocompatible, hydrophilic polymer that is positively charged at a first pH and in electronically neutral form at a higher pH; and administering an effective amount of the beneficial agent into the vaginal passage when the pH is equal to or above 7.

The present invention also provides for a method of contraception in a female individual, comprising: positioning in the vaginal passage of the female individual prior to sexual intercourse a pH-responsive film for the administration of a beneficial agent effective to prevent pregnancy, wherein the pH-responsive film comprises an effective amount of the beneficial agent; and a biocompatible, hydrophilic polymer that is positively charged at a first pH and in electronically neutral form at a higher pH; and administering an effective amount of the beneficial agent into the vaginal passage the pH is equal to or above 7. The beneficial agent may be an ionizable agent, such that an increase in the local pH neutralizes the hydrophilic polymer.

The present pH-responsive films are neater then the currently available foams, suppositories, gels, and creams, provide no systemic side-effects, and completely degrade. Thus, the films satisfy the need in the art for an easy to use, non-messy, and, most importantly, effective product that will prevent unwanted pregnancies and/or prevent the transmission of STDs including HIV. Moreover, the film is inexpensive to make, unobtrusive to the user, and non-irritating to both partners.

The following examples are merely illustrative of the present invention, and they should not be considered as limiting the scope of the invention in any way, as these examples and other equivalents thereof will become apparent to those versed in the art in the light of the present disclosure and the accompanying claims.

### EXAMPLES

The following materials were used in formulating the films of different compositions: chitosan lactate (supplied by Vanson/Halo source); Pluronic 108 (supplied by BASF); HPMC 50 (supplied by Dow Chemicals); D,L-lactic acid (supplied by Aldrich); citric acid (supplied by Mallinckrodt), and glycerin (supplied by Sigma).

### Example 1

### Film Preparation

Stock solutions of chitosan lactate (4%), Pluronic 108 (4%) and HPMC 50 (10%) were prepared in water. As specified in the following examples, particular amounts of chitosan lactate solution were mixed with the solution of Pluronic 108 under high shear. Subsequently, HPMC 50 solution was added and mixed thoroughly. Depending on the formulation, lactic acid, citric acid, PVP90, PEG 400, and/or glycerine were added to the solutions of the polymer. Materials were obtained from the following sources: Chitosan lactate (Vanson/Halo); Pluronic 108 (BASF); HPMC 50 (Dow Chemical Co.); DL lactic acid (Aldrich Chemical Co.); Citric acid (Mallinckrodt); Polyvinyl pyrrolidone 90 (PVP90, BASF); Polyethylene glycol 400 (PEG 400, BASF).

The polymer solutions were cast on to a 3 mil Melinex polyester sheet using a casting knife. The thickness of the wet film was 4 mil. The film was air dried overnight and then in a vacuum oven at 30° C. for 2 hours. The different compositions that were made are as follows:

Weight Percent in dried film

| | Chitosan | | | |
|---|---|---|---|---|
| Formulation | Lactate | Pluronic 108 | Glycerin | Total |
| 14169-4-1 | 100 | 0 | 0 | 100 |
| 14169-4-2 | 44.93 | 11.02 | 44.05 | 100 |
| 14169-4-3 | 43.3 | 15.46 | 41.24 | 100 |
| 14169-4-4 | 48.04 | 5.77 | 46.19 | 100 |
| 14169-4-5 | 65.53 | 6.14 | 28.33 | 100 |
| 14169-4-6 | 91.43 | 8.57 | 0 | 100 |

All of the above films were transparent and demonstrated good flexibility.

| | Weight Percent in dried film | | | | |
|---|---|---|---|---|---|
| | Chitosan | | | | |
| | Lactate | HPMC 50 | Pluronic 108 | | |
| Formulations | (4%) | (10%) | (4%) | Glycerin | Total |
| 14169-61-1 | 38.41 | 24.12 | 0 | 37.47 | 100 |
| 14169-61-2 | 35.44 | 21.86 | 8.54 | 34.16 | 100 |
| 14169-61-3 | 31.94 | 26.23 | 11.41 | 30.42 | 100 |
| 14169-61-4 | 36.72 | 23.45 | 4.43 | 35.4 | 100 |

| | Weight Percent in dried film | | | | | |
|---|---|---|---|---|---|---|
| | Chitosan | Pluronic | | DL-Lactic | Gly- | |
| Formulations | Lactate | 108 | HPMC 50 | acid | cerin | Total |
| 14169-36-1 | 27.65 | 3.22 | 44.8 | 24.33 | 0 | 100 |
| 14169-39-2 | 38.18 | 3.67 | 31.28 | 0 | 26.87 | 100 |

| | Weight Percent in dried film | | | | | |
|---|---|---|---|---|---|---|
| | Chitosan | Pluronic | DL-Lactic | | PEG | |
| Formulations | Lactate | 108 | acid | PVP90 | 400 | Total |
| 14169-43-1 | 46.04 | 5.34 | 40.52 | 5.15 | 2.95 | 100 |
| 14169-43-2 | 26.35 | 3.05 | 28.45 | 26.97 | 15.18 | 100 |

| | Weight Percent in dried film | | | | | |
|---|---|---|---|---|---|---|
| | Chitosan | Pluronic | HPMC | Lactic | Citric | |
| Formulations | Lactate | 108 | 50 | acid | Acid | Total |
| 14169-51-1 | 27.62 | 3.31 | 44.75 | 0 | 24.32 | 100 |
| 14169-51-2 | 40 | 30 | 0 | 25 | 5 | 100 |
| 14169-51-3 | 40 | 30 | 0 | 0 | 30 | 100 |
| 14169-50-3 | 27.77 | 3.33 | 44.45 | 18.33 | 6.12 | 100 |

The physical observation and dissolution behavior of the above films were evaluated in three different media—water, simulated vaginal fluid (pH 4.0), and MHF buffer (pH 7.5). As can be noted from the table below the dissolution properties of the film can be easily varied depending on the requirements.

| | Weight Percent | | | | | |
|---|---|---|---|---|---|---|
| | | | | DL- | | |
| | Chitosan | Pluronic | HPMC | Lactic | | Citric |
| Formulations | Lactate | 108 | 50 | acid | Glycerin | Acid |

| | | | | | | |
|---|---|---|---|---|---|---|
| 14169-4-1 | 100 | 0 | 0 | 0 | 0 | 0 |
| 14169-4-2 | 44.93 | 11.02 | 0 | 0 | 44.05 | 0 |
| 14169-4-3 | 43.3 | 15.46 | 0 | 0 | 41.24 | 0 |
| 14169-4-4 | 48.04 | 5.77 | 0 | 0 | 46.19 | 0 |
| 14169-1-1 | 80.5 | 19.45 | 0 | 0 | 0 | 0 |
| 14169-5-1 | 0 | 3.01 | 75.38 | 0 | 21.61 | 0 |
| 14169-5-2 | 0 | 4.4 | 78.022 | 0 | 17.58 | 0 |
| 14169-5-3 | 0 | 6.25 | 78.12 | 0 | 15.62 | 0 |
| 14169-19-1 | 11.11 | 2.67 | 66.67 | 0 | 19.55 | 0 |
| 14169-19-2 | 20 | 3.2 | 53.33 | 0 | 23.47 | 0 |
| 14169-19-3 | 27.62 | 3.32 | 44.75 | 0 | 24.31 | 0 |
| 14169-19-4 | 38.18 | 3.67 | 31.28 | 0 | 26.87 | 0 |
| 14169-19-5 | 80.55 | 19.35 | 0 | 0 | 0 | 0 |
| 14169-36-1 | 27.65 | 3.21 | 44.8 | 24.33 | 0 | 0 |
| 14169-39-1 | 20 | 3.2 | 53.33 | 0 | 23.47 | 0 |
| 14169-39-2 | 38.18 | 3.67 | 31.28 | 0 | 26.87 | 0 |
| 14169-39-3 | 27.65 | 3.22 | 44.8 | 24.33 | 0 | 0 |
| 14169-50-1 | 38.1 | 33.33 | 0 | 23.81 | 0 | 4.76 |
| 14169-50-2 | 40 | 30 | 0 | 25 | 0 | 5 |
| 14169-50-3 | 27.77 | 3.33 | 44.45 | 18.33 | 0 | 6.12 |
| 14169-51-1 | 27.62 | 3.31 | 44.75 | 0 | 0 | 24.32 |
| 14169-51-2 | 40 | 30 | 0 | 25 | 0 | 5 |
| 14169-51-3 | 40 | 30 | 0 | 0 | 0 | 30 |

| | Water | | | Physical |
|---|---|---|---|---|
| Formulations | Solubility | MHF Buffer | SVF, pH 4.0 | Observation |
| 14169-4-1 | gel, pH = 3.86 | hydrogel film, 6.3 | gel, 4.04 | clear, yellow, |
| | | | | flexible |
| 14169-4-4 | film, pH 3.25 | 6.61, film | uniform film, | pliable, clear, yellow |
| | | | 3.71 | |
| 14169-5-1 | yes, pH 7.00 | — | dissolved, 3.67 | clear, semi-flexible |
| 14169-5-2 | yes, pH 6.77 | — | dissolved, 3.60 | translucent, semi- |
| | | | | flexible |
| 14169-5-3 | yes, pH 6.97 | — | dissolved, 3.81 | translucent, semi- |
| | | | | flexible |
| 14169-5-4 | yes, pH 6.32 | — | dissolved, 3.70 | clear, semi-flexible |
| 14169-19-1 | fragmented film, | 7.25, film | uniform film, | flexible, clear yellow |
| | pH2.77 | | 3.67 | |
| 14169-19-2 | fragmented film, | 7.00, film | uniform film, | flexible, clear yellow |
| | pH2.88 | | 3.85 | |
| 14169-19-3 | film, pH 3.98 | 6.86, film | uniform film, | flexible, clear yellow |
| | | | 3.87 | |
| 14169-19-4 | film, pH 2.96 | 6.28, gel | uniform film, | flexible, clear yellow |
| | | | 3.96 | |
| 14169-19-5 | gel, pH 4.91 | | gel, 3.95 | flexible, clear yellow |
| 14169-36-1 | gel, film, pH | dissolve, pH 4.21 | dissolved, 3.46 | translucent, semi- |
| | 2.54 | | | flexible, pale yellow |
| 14169-39-1 | film, pH 2.90 | 7.00, film | film, 3.92 | flexible, clear yellow |
| 14169-39-2 | film, gel pH | 6.82, film | film, 3.82 | pliable, clear, yellow |
| | 2.93 | | | |
| 14169-39-3 | gel, pH 2.57 | 4.11, dissolved | gel, 3.47 | flexible, clear yellow |
| 14169-50-1 | yes, pH 2.21 | low viscous, 3.91 | dissolved, 3.25 | translucent, pale |
| | | | | yellow, brittle |
| 14169-50-2 | yes, pH 2.76 | viscous, 3.70 | dissolved, 3.27 | translucent, pale |
| | | | | yellow, brittle |
| 14169-50-3 | yes, pH 1.94 | gel, 3.77 | Dissolved, 3.31 | translucent, semi- |
| | | | | flexible, pale yellow |
| 14169-50-4 | yes, pH 2.08 | dissolved, 3.78 | dissolved, 3.12 | pliable, clear, pale |
| | | | | yellow |
| 14169-51-1 | yes, pH 2.84 | dissolved, 3.64 | dissolved, 3.05 | pliable, translucent, |
| | | | | pale yellow |
| 14169-51-2 | yes, pH 2.25 | film, 3.94 | film in pieces, | semi flexible, |
| | | | 3.33 | translucent, pale |
| | | | | yellow |

### Example 2

### Evaluation of Films In Sperm Motility Assay

Sperm Isolation

Male Sprague-Dawley-rats, between the ages of 16-22 weeks, were used for this study. Following anesthesia, both testes were removed and the epididymides were collected, rinsed in warm Dulbecco's phosphate buffered saline (37° C.), and placed in a petri dish containing approximately 10 mL warm MHF-10 (37° C.). The epididymides were minced with scissors to release sperm. Pipette sperm suspension into a 50 mL plastic centrifuge tube with a screw cap. Dilute sperm with additional MHF-10 (approximately 5-12 mL) to form a milky suspension. The initial percentage of motile sperm must be at least 70% for a valid assay, and is determined by inoculating 200 μL sperm suspension into 0.8 mL MHF-10 and counting motile versus nonmotile sperm.

In Vitro Exposure

After the test article concentrations equilibrated in a 36° to 37° C. incubator for at least 30 minutes, 200 μL aliquots of the sperm suspension were added to the 0.8 mL test compound concentrations, two at a time, at 5 minute intervals, beginning with the highest concentration and proceeding to in order to the lowest concentration, then ending with vehicle and positive controls. The final volume was 1 mL per test compound concentration.

Data Collection

Motility was assessed using a microscope. 50-μL aliquots of the test concentration, containing sperm, were added to the wells of preheated glass slides and covered with preheated glass coverslips. Pipette tips, slides, and coverslips were maintained at 36-37° C. on a warming plate. Motility was assessed at the appropriate exposure times, generally 20 and 40 min after addition of sperm to the test concentration. The degrees of motility of≧200 sperm per concentration per time point were evaluated as either not motile (0), incipiently motile (quivering/pulsing/wiggling) (1), slowly motile (2), or rapidly motile (3).

Evaluation of Data

Criteria for a Valid Assay. The data from this assay was considered acceptable if the following conditions were met: 1) the initial motility of the sperm in MHF-10 was at least 70%; 2) the motility of the vehicle control culture did not drop below 30% at the late time point; and 3) the positive control concentration yielded nonmotile sperm at the 20 min time point. Percent motility in the tables below is calculated as (Prog. Motile)/Total (Prog.+Incip.+Non)×100.

The following compositions were evaluated in the sperm motility assay and the results are as follows:

Sperm Study

| No. | Formulations | Weight (mg) | Thickness (mil) |
|---|---|---|---|
| 1 | Chitosan Lactate | 4.3 | 1 |
| | (14169-4-1) | | |
| 2 | 14169-19-5 | 7.1 | 1 |
| 3 | 14169-19-4 | 13.3 | 4 |
| 4 | 14169-19-2 | 16.7 | 4 |
| 5 | 14169-5-3 | 19.8 | 4 |

| | | Weight Percent | | | |
|---|---|---|---|---|---|
| | | Chitosan | Pluronic | HPMC | |
| No. | Formulations | Lactate | 108 | 50 | Glycerin |
| 1 | 14169-4-1 | 100 | 0 | 0 | 0 |
| 2 | 14169-19-5 | 80.65 | 19.35 | 0 | 0 |
| 3 | 14169-19-4 | 38.18 | 3.67 | 31.28 | 26.87 |
| 4 | 14169-19-2 | 20 | 3.2 | 53.33 | 23.47 |
| 5 | 14169-5-3 | 0 | 7.41 | 74.07 | 18.52 |

Results

| Time | 1 min | | | |
|---|---|---|---|---|
| Test Agent | Non-Mot | Incip.Mot | Prog-Motile | % Motility |
| 1 | 62 | 11 | 31 | 29.8 |
| 2 | 38 | 20 | 43 | 42.6 |
| 3 | 49 | 25 | 48 | 39.3 |
| 4 | 26 | 26 | 58 | 52.7 |
| 5 | 56 | 51 | 19 | 15.1 |
| Control | 21 | 16 | 64 | 63.3 |
| N-9 | 100 | 0 | | 0 |

| Time | 15 min | | | |
|---|---|---|---|---|
| Test Agent | Non-Motile | Incip.Mot | Prog-Motile | % Motility |
| 1 | 72 | 25 | 17 | 14.9 |
| 2 | 36 | 31 | 52 | 43.7 |
| 3 | 39 | 22 | 48 | 44.0 |
| 4 | 68 | 24 | 11 | 10.7 |
| 5 | 83 | 38 | 17 | 12.3 |
| Control | 39 | 19 | 61 | 51.3 |
| N-9 | 100 | 0 | 0 | 0 |

| Time | 30 min | | | |
|---|---|---|---|---|
| Test Agent | Non-Motile | Incip.Mot | Prog-Motile | % Motility |
| 1 | 81 | 20 | 8 | 7.3 |
| 2 | 75 | 14 | 17 | 16.0 |
| 3 | 94 | 28 | 0 | 0 |
| 4 | 51 | 25 | 36 | 32.1 |
| 5 | 80 | 19 | 20 | 16.8 |
| Control | 39 | 28 | 39 | 36.8 |
| N-9 | 100 | 0 | 0 | 0 |

### Experiment B

In this experiment, chitosan lactate solution, lactic acid solution, and films having different amounts of chitosan lactate and/or lactic acid were evaluated for their abilities to reduce sperm motility. The films were prepared as described above.

| No | Formulations | Weight (mg) | Thickness (mil) |
|---|---|---|---|
| 1 | Chitosan Lactate 4% | 200 | solution |
| 2 | Lactic Acid 4% | 200 | solution |
| 3 | 14169-19-4 | 50 | 3 |
| 4 | 14169-19-2 | 49.1 | 6 |
| 5 | 14169-36-1 | 49.4 | 3 |

| | | Weight Percent | | | | |
|---|---|---|---|---|---|---|
| | | Chitosan | Pluronic | HPMC | DL-Lactic | |
| No | Formulations | Lactate | 108 | 50 | acid | Glycerin |
| 3 | 14169-19-4 | 38.18 | 3.67 | 31.28 | 0 | 26.87 |
| 4 | 14169-19-2 | 20 | 3.2 | 53.33 | 0 | 23.47 |
| 5 | 14169-36-1 | 27.65 | 3.21 | 44.8 | 24.33 | 0 |

Results

| | 1 min | | | | |
|---|---|---|---|---|---|
| Time | Volume (μl) | Prog.-Mot | Incip.Mot | Non-Mot | % Motility |
| Control | — | 160 | 25 | 48 | 68.7 |
| 1 | 200 | 69 | 29 | 104 | 34.1 |
| 1 | 400 | 62 | 29 | 129 | 28.2 |
| 1 | 800 | 21 | 12 | 169 | 10.4 |
| 2 | 100 | 62 | 41 | 103 | 30.1 |
| 2 | 200 | 0 | 0 | 200 | 0 |
| 3 | 50 mg of film | 76 | 26 | 102 | 37.3 |
| 4 | 49.1 mg | 84 | 19 | 97 | 42.0 |
| 5 | 49.4 mg | 0 | 0 | 200 | 0 |
| N9 | | 0 | 0 | 200 | 0 |

| | 15 min | | | | |
|---|---|---|---|---|---|
| Time | Volume (μl) | ProgMot | Incip.Mot | Non-Mot | % Motility |
| Control | | 126 | 27 | 59 | 59.4 |
| 1 | 200 | 57 | 12 | 138 | 27.5 |
| 1 | 400 | 7 | 8 | 198 | 3.3 |
| 1 | 800 | 0 | 0 | 200 | 0 |
| 2 | 100 | 44 | 20 | 142 | 21.4 |
| 2 | 200 | 0 | 0 | 200 | 0 |
| 3 | 50 mg of Film | 4 | 30 | 170 | 2.0 |
| 4 | 49.1 mg | 42 | 15 | 146 | 20.7 |
| 5 | 49.4 mg | 0 | 0 | 200 | 0 |
| N9 | | | 0 | 200 | 0 |

| | 30 min | | | | |
|---|---|---|---|---|---|
| Time | Volume (μl) | Prog.Mot | Incip.Mot | Non-Mot | % Motility |
| Control | — | — | — | — | — |
| 1 | 200 | 106 | 20 | 95 | 48.0 |
| 1 | 400 | 21 | 2 | 180 | 10.3 |
| 1 | 800 | 0 | 1 | 200 | 0 |
| 2 | 100 | 38 | 16 | 157 | 18.0 |
| 2 | 200 | 0 | 0 | 200 | 0 |
| 3 | 50 mg of film | 0 | | | 0 |
| 4 | 49.1 mg | 0 | 15 | 189 | 0 |
| 5 | 49.4 mg | 30 | 10 | 166 | 14.6 |
| N9 | | 0 | 0 | 200 | 0 |

Count done in suspension of 1.6 ml of MHF-10 + 0.1 ml sperm suspension + test agent

As can be seen from the data above, formulation 5 (14169-36-1 film) kills sperm in 1 min. As the volume of chitosan lactate solution increases (200 to 800 microliters), the number of non-motile sperm increased from 104 to 169 in 1 min. Additionally, as the volume of critic acid increases (100 to 200 microliters), the number of non-motile sperm increased from 103 to 200 in 1 min.

### Experiment C

This experiment provides an evaluation of film weight relative to sperm kill. Films from the indicated formulations.

| | Weight Percent | | | | |
|---|---|---|---|---|---|
| | Chitosan | Pluronic | | DL-Lactic | |
| Formulations | Lactate | 108 | HPMC 50 | acid | Glycerin |
| 14169-36-1 | 27.65 | 3.21 | 44.8 | 24.33 | 0 |
| 14169-39-2 | 38.18 | 3.67 | 31.28 | 0 | 26.87 |
| 14169-39-3 | 27.65 | 3.22 | 44.8 | 24.33 | 0 |

| No. | Formulations | Weight (mg) | Thickness (mil) |
|---|---|---|---|
| 1 | 14169-39-3 | 25.5 | 4 |
| 2 | 14169-36-1 | 25.2 | 4 |
| 3 | 14169-39-2 | 24.9 | 4 |
| 4 | VCF | 25.1 | 4 |
| 5 | 14169-39-3 | 50.2 | 4 |
| 6 | 14169-36-1 | 49.9 | 4 |
| 7 | 14169-39-2 | 51.0 | 4 |
| 8 | VCF | 50.5 | 4 |

Results

| Time | 1 min | | | |
|---|---|---|---|---|
| Test Agent | Prog-Mot | Incip.Mot | Non-Mot | % Motility |
| Control | 135 | 25 | 62 | 60.8 |
| 1 | 22 | 34 | 150 | 10.7 |
| 2 | 8 | 65 | 152 | 3.6 |
| 3 | 45 | 38 | 120 | 22.2 |
| 4 | 0 | 0 | 200 | 0 |
| 5 | 0 | 0 | 200 | 0 |
| 6 | 0 | 0 | 200 | 0 |
| 7 | 0 | 111 | 80 | 0 |
| 8 | 0 | 0 | 200 | 0 |
| N9 | 0 | 0 | 200 | 0 |

| | |
|---|---|
| Time | 15 min |

| Test Agent | Mot | Incip.Mot | Non-Mot | Total |
|---|---|---|---|---|
| Control | 118 | 22 | 69 | 56.4 |
| 1 | 0 | 10 | 190 | 0 |
| 2 | 0 | 0 | 200 | 0 |
| 3 | 0 | 34 | 170 | 0 |
| 4 | 0 | 0 | 200 | 0 |
| 5 | 0 | 0 | 200 | 0 |
| 6 | 0 | 0 | 200 | 0 |
| 7 | 0 | 18 | 157 | 0 |
| 8 | 0 | 0 | 200 | 0 |
| N9 | 0 | 0 | 200 | 0 |

| Time | 30 min | | | |
|---|---|---|---|---|
| Test Agent | Mot | Incip.Mot | Non-Mot | Total |
| Control | 90 | 50 | 65 | 43.9 |
| 1 | 0 | 0 | 200 | 0 |
| 2 | 0 | 0 | 200 | 0 |
| 3 | 0 | 22 | 180 | 0 |
| 4 | 0 | 0 | 200 | 0 |
| 5 | 0 | 0 | 200 | 0 |
| 6 | 0 | 0 | 200 | 0 |
| 7 | 0 | 0 | 200 | 0 |
| 8 | 0 | 0 | 200 | 0 |
| N9 | 0 | 0 | 200 | 0 |

As can be seen from the data above, the 50 mg formulations of each of 14169-39-3 and 14169-36-1 provided complete sperm kill (non-motile) in 1 min. Similar efficacy was found for the 25 mg formulations although portions of the sperm were found to be incipient motile (not dead, but rather inactive).

### Experiment D

The experiment illustrates the spermicidal activity of lactic acid and citric acid, in combination with the indicated films. Again, the films were prepared as described above.

Sperm Study

| No | Formulations | Weight (mg) | Thickness (mil) |
|---|---|---|---|
| 1 | VCF | 50.09 | 4 |
| 2 | 14169-51-3 | 50.0 | 4 |
| 3 | 14169-50-3 | 50.09 | 4 |
| 4 | 14169-36-1 | 50 | 4 |

| | | Chitosan | Pluronic | HPMC | Lactic | Citric |
|---|---|---|---|---|---|---|
| No | Formulations | Lactate | 108 | 50 | Acid | Acid |
| 2 | 14169-51-3 | 40 | 30 | 0 | 0 | 30 |
| 3 | 14169-50-3 | 27.77 | 3.33 | 44.45 | 18.33 | 6.12 |
| 4 | 14169-36-1 | 27.65 | 3.21 | 44.8 | 4.33 | 0 |

Results

| Time | 1 min | | | |
|---|---|---|---|---|
| Test Agent | Prog.Mot | Incip.Mot | Non-Mot | % Motility |
| Control | 125 | 40 | 35 | 62.5 |
| 1 | 0 | 0 | 200 | 0 |
| 2 | 0 | 0 | 200 | 0 |
| 3 | 0 | 0 | 200 | 0 |
| 4 | 0 | 0 | 200 | 0 |
| N9 | 0 | 0 | 200 | 0 |

| Time | 15 min | | | |
|---|---|---|---|---|
| Test Agent | Prog.Mot | Incip.Mot | Non-Mot | % Motility |
| Control | 100 | 50 | 50 | 50 |
| 1 | 0 | 0 | 200 | 0 |
| 2 | 0 | 0 | 200 | 0 |
| 3 | 0 | 0 | 200 | 0 |
| 4 | 0 | 0 | 200 | 0 |
| N9 | 0 | 0 | 200 | 0 |

| Time | 30 min | | | |
|---|---|---|---|---|
| Test Agent | Mot | Incip.Mot | Non-Mot | % Motility |
| Control | 80 | 50 | 70 | 40 |
| 1 | 0 | 0 | 200 | 0 |
| 2 | 0 | 0 | 200 | 0 |
| 3 | 0 | 0 | 200 | 0 |
| 5 | 0 | 0 | 200 | 0 |
| N9 | 0 | 0 | 200 | 0 |

In each instance above, the films provided complete spermicidal activity within 1 min.

### Example 3

### Evaluation of Films Against Gram Positive and Gram Negative Bacteria

Two polymers, 14169-39-2 and 14169-39-3, were tested for antimicrobial activity against Escherichia coli ATCC strain 25922, and against Staphylococcus aureus ATCC strain 25923. The details of the formulations are as follows:

| | Weight (g) | | | | | |
|---|---|---|---|---|---|---|
| | Chitosan | Pluronic | HPMC | Gly- | Lactic | |
| Sample no. | Lactate | 108 | 50 | cerin | Acid | Total (g) |
| 14169-39-2 | 6.25 | 0.6 | 5.12 | 4.4 | 0 | 16.37 |
| 14169-39-3 | 2.5 | 0.29 | 4.05 | 0 | 2.2 | 6.84 |

| | Weight Solution (g) | | | | | |
|---|---|---|---|---|---|---|
| | Chitosan | | | | | |
| | Lactate | Pluronic | HPMC | Gly- | Lactic | |
| Sample no. | (4%) | 108 | 50 | cerin | Acid | Total (g) |
| 14169-39-2 | 156.25 | 15 | 51.2 | 4.4 | 0 | 226.85 |
| 14169-39-3 | 62.5 | 7.4 | 40.5 | 0 | 2.2 | 110.4 |

| | Weight Percent | | | | | |
|---|---|---|---|---|---|---|
| | Chitosan | Pluronic | HPMC | Gly- | Lactic | |
| Sample no. | Lactate | 108 | 50 | cerin | Acid | Total (g) |
| 14169-39-2 | 38.18 | 3.67 | 31.28 | 26.87 | 0 | 100 |
| 14169-39-3 | 27.65 | 3.22 | 44.8 | 0 | 24.33 | 100 |

Experimental Procedure

Two grams of each formulation were weighed out using aseptic techniques. To each formulation sterile Mueller-Hinton broth (MBH) (18 mL) was added. This constituted a 1:10 dilution (the dilution factor was actually slightly less than 1:10 since 2 grams of each of the formulations represent slightly less than a 2 mL volume). A second 1:10 dilution was made by transferring 0.5 mL to 4.5 mL sterile MBH medium. This constituted a 1:100 dilution.

For each formulation, a 2×3 tube was prepared by transferring 4 mL of the 1:10 and 1:100 dilutions to sterile glass test tubes as indicated below. One tube each for the negative controls (MHB and bacteria only) was prepared.

The tubes were inoculated as indicated, except the sterility control tubes, with E. coli and S. aureus bacteria. The inoculum size was about 106 bacteria/mL. The tubes were incubated for 24 hours at 35° C. Each tube was visually inspected for growth (turbidity).

Results and Discussion

Polymer 14169-2(2)0:

E. coli: there was apparent growth in the 1:100 tube. However this needed to be confirmed as there was a fair amount of precipitate in the sterility control tube. There was also a slight amount of precipitate in the 1:10 sterility control tube. Therefore it could not be determined if the slight turbidity that was observed in the 1:10 was due to bacterial growth.

To distinguish between precipitate and growth (turbidity), a loopful of the 1:10 and 1:100 tubes was streaked on nutrient agar plates. A loopful from the sterility control tubes was also streaked on nutrient agar plates. After overnight incubation at 35° C., the plates were inspected for bacterial growth. Growth was observed only on the plates that were streaked with a loopful of the 1:100 dilutions. The results indicate that the polymer exerts antimicrobial activity against E. coli when it is diluted to 1:10. When diluted 1:100, the polymer is present at a concentration that is too low to exert antimicrobial activity. There was good growth in the negative control tubes (medium and bacteria), indicating that the growth medium adequately supported the growth of E. coli.

S. aureus. The results were identical to those obtained with E. coli.

### Example 14169-39-3:

E. coli: as with 14169-39-2, there was apparent growth in the 1:100 tube. However this needed to be confirmed as there was a fair amount of precipitate in the sterility control tube. In contrast to 14169-39-2, there was no precipitate in the 1:10 sterility control tube, and there was no growth observed in the 1:10 tube with E. coli.

To distinguish between precipitate and growth (turbidity), a loopful of the 1:100 tube was streaked on nutrient agar plates. For consistency with what was done with polymer 2, a loopful of the 1:10 tube containing E. coli was also streaked on an agar plate. After overnight incubation at 35° C. the plates were inspected for bacterial growth. Growth was observed only on the plates that were streaked with a loopful of the 1:100 dilution. The results indicate that the polymer exerts antimicrobial activity against E. coli when it is diluted 1:10. When diluted 1:100 the polymer is present at a concentration that is too low to exert antimicrobial activity.

S. aureus. The results were identical to those obtained with E. coli.

The results are summarized below:

| TEST ORGANISM | 14169-39-2 | 14169-39-3 |
|---|---|---|
| E.COLI | 1:10 No growth | 1:10 No growth |
| | 1:100 Growth | 1:100 Growth |
| S. AUREUS | 1:10 No growth | 1:10 No growth* |
| | 1:100 Growth | 1:100 Growth |

### Example 4

This example illustrates film compositions containing a benzoic acid component, as illustrative of an acidic therapeutic agent. For the films below,

| | Weight (g) | | | | | | |
|---|---|---|---|---|---|---|---|
| | Chitosan | Pluronic | HPMC | | Lactic | Benzoic | |
| Sample no. | Lactate | 108 | 50 | Glycerin | Acid | Acid | Total (g) |
| 14169-39-2 | 6.25 | 0.6 | 5.12 | 4.44 | 0 | 0.05 | 16.46 |
| (87) | | | | | | | |
| 14169-39-3 | 2.5 | 0.3 | 4.05 | 0 | 2.2 | 0.0331 | 9.083 |
| (87) | | | | | | | |
| 14169-5-4 | 0 | 2 | 20 | 5.08 | 0 | 0.0889 | 27.17 |
| (87) | | | | | | | |

| | Weight Solution (g) | | | | | | |
|---|---|---|---|---|---|---|---|
| | Chitosan | | | | | | |
| | Lactate | Pluronic | HPMC | | Lactic | Benzoic | |
| Sample no. | (4%) | 108 | 50 | Glycerin | Acid | acid | Total (g) |
| 14169-39-2 | 156.25 | 15 | 51.2 | 4.4 | 0 | 0.05 | 226.9 |
| (87) | | | | | | | |
| 14169-39-3 | 62.5 | 7.4 | 40.84 | 0 | 2.2 | 0.331 | 113.27 |
| (87) | | | | | | | |
| 14169-5-4 | 0 | 50 | 200 | 5.08 | 0 | 0.0889 | 255.17 |
| (87) | | | | | | | |

| | Weight Percent | | | | | | |
|---|---|---|---|---|---|---|---|
| | Chitosan | Pluronic | HPMC | | Lactic | Benzoic | |
| Sample no. | Lactate | 108 | 50 | Glycerin | Acid | Acid | Total (g) |
| 14169-39-2 | 37.97 | 3.645 | 31.105 | 26.974 | 0 | .3037 | 100 |
| (87) | | | | | | | |
| 14169-39-3 | 27.52 | 3.3 | 44.6 | 0 | 24.22 | 0.36 | 100 |
| (87) | | | | | | | |
| 14169-5-4 | 0 | 7.36 | 73.61 | 18.7 | 0 | 0.33 | 100 |
| (87) | | | | | | | |

| | Weight (g) | | | | | | | |
|---|---|---|---|---|---|---|---|---|
| Sample | Chitosan | Pluronic | HPMC | | | Lactic | Benzoic | Total |
| no. | Lactate | 108 | 50 | Polyox K 60 | Glycerin | Acid | Acid | (g) |
| 14169- | 3.125 | 0.3 | 1.807 | 0.833 | 2.22 | 0 | 0.0279 | 8.3129 |
| 39-2 | | | | | | | | |
| (89) | | | | | | | | |
| 14169- | 2.5 | 0.3 | 2.877 | 1.213 | 0 | 2.2503 | 0.035 | 9.175 |
| 39-3 | | | | | | | | |
| (89) | | | | | | | | |

| | Weight Solution (g) | | | | | | | |
|---|---|---|---|---|---|---|---|---|
| | Chitosan | Pluronic | HPMC | | | | | |
| Sample | Lactate | 108 | 50 | Polyox K 60 | Glycerin | Lactic | Benzoic | Total |
| no. | (4%) | (4%) | (10%) | (5%) | (g) | Acid | Acid | (g) |
| 14169- | 78.125 | 7.5 | 18.07 | 16.67 | 2.2 | 0 | 0.0279 | 122.5929 |
| 39-2 | | | | | | | | |
| (89) | | | | | | | | |
| 14169- | 62.5 | 7.4 | 28.77 | 24.26 | 0 | 2.2508 | 0.035 | 125.22 |
| 39-3 | | | | | | | | |
| (89) | | | | | | | | |

| | Weight Percent | | | | | | | |
|---|---|---|---|---|---|---|---|---|
| Sample | Chitosan | Pluronic | | | | Lactic | Benzoic | Total |
| no. | Lactate | 108 | HPMC 50 | Polyox K 60 | Glycerin | Acid | Acid | (g) |
| 14169- | 37.59 | 3.61 | 21.74 | 10.02 | 26.71 | 0 | 0.34 | 100 |
| 39-2 | | | | | | | | |
| (89) | | | | | | | | |
| 14169- | 27.25 | 3.27 | 31.36 | 13.22 | 0 | 24.53 | 0.38 | 100 |
| 39-3 | | | | | | | | |
| (89) | | | | | | | | |

What is claimed is:

1. A pH-responsive film comprising:
(a) a biocompatible, hydrophilic polymer that is positively charged at a first pH and in electronically neutral form at a higher pH; and
(b) an alkylene oxide polymer or copolymer.

2. A pH-responsive film of claim 1, wherein said biocompatible, hydrophilic polymer is chitosan and said alkylene oxide polymer or copolymer is selected from the group consisting of poly(alkylene oxides) and poloxamer copolymers.

3. A pH-responsive film of claim 2, wherein said alkylene oxide polymer or copolymer is a poloxamer copolymer.

4. A pH-responsive film of claim 3, wherein said poloxamer copolymer is selected from the group consisting of Pluronic® 108 and Pluronic®127.

5. A pH-responsive film of claim 4, further comprising a cellulose ether selected from the group consisting of hydroxyethyl cellulose, hydroxypropyl cellulose and hydroxypropyl methylcellulose.

6. A pH-responsive film of claim 5, wherein said cellulose ether is hydroxypropyl methyl cellulose.

7. A pH-responsive film of claim 4, further comprising glycerin.

8. A pH-responsive film of claim 6, further comprising glycerin.

9. A pH-responsive film of claim 7, further comprising DL-lactic acid.

10. A pH-responsive film of claim 8, further comprising DL-lactic acid.

11. A pH-responsive film of claim 1, comprising of from 20 to 60 weight percent chitosan lactate, of from 3 to 35 weight percent of a poloxamer copolymer, of from 5 to 45 weight percent of hydroxypropyl methylcellulose, and of from 5 to 45 weight percent glycerin.

12. A pH-responsive film for administration of a beneficial agent, comprising:
(a) an effective amount of a beneficial agent;
(b) a biocompatible, hydrophilic polymer that is positively charged at a first pH and in electronically neutral form at a higher pH; and
(c) an alkylene oxide polymer or copolymer.

13. The film of claim 12, said biocompatible, hydrophilic polymer is chitosan and said alkylene oxide polymer or copolymer is selected from the group consisting of poly(alkylene oxides) and poloxamer copolymers.

14. The film of claim 13, wherein said alkylene oxide polymer or copolymer is a poloxamer copolymer.

15. The film of claim 14, wherein said poloxamer copolymer is selected from the group consisting of Pluronic® 108 and Pluronic® 127.

16. The film of claim 15, further comprising a cellulose ether selected from the group consisting of hydroxyethyl cellulose, hydroxypropyl cellulose and hydroxypropyl methylcellulose.

17. The film of claim 16, wherein said cellulose ether is hydroxypropyl methylcellulose.

18. The film of claim 12, wherein the biocompatible hydrophilic polymer has a pKa≦6.

19. The film of claim 18, wherein the biocompatible hydrophilic polymer is bioerodible.

20. The film of claim 19, wherein the biocompatible hydrophilic polymer is water swellable.

21. The film of claim 20, wherein the biocompatible hydrophilic polymer is bioadhesive.

22. The film of claim 12, wherein the beneficial agent is an acid.

23. The film of claim 22, wherein the pKa of the beneficial agent is≧3.

24. The film of claim 23, wherein the pKa of the beneficial agent is about 3.

25. The film of claim 22, wherein the acid contains at least two moieties selected from carboxylic acids, sulfonic acids, phosphonic acids, and mixtures thereof.

26. The film of claim 22, wherein the acid is an organic acid.

27. The film of claim 12, wherein at the first pH the biocompatible hydrophilic polymer comprises a salt formed with the organic acid.

28. The film of claim 27, further comprising excess organic acid.

29. The film of claim 26, wherein the organic acid is monomeric.

30. The film of claim 29, wherein the monomeric organic acid has the structural formula [R—(Lₓ—OOH)_{y}]_{z} wherein:
R is selected from C₁-C₁₂ alkyl, C₂-C₁₂ alkenyl, C₅-C₁₆ aryl, and C₅-C₁₆ heteroaryl (including substituted such moieties);
L is C₁-C₈ alkylene or C₂-C₈ alkenylene;
x is 0 or 1;
y is an integer in the range of 2 to 8 inclusive; and
z is 1, 2 or 3,
with the proviso that if z is 2 or 3, the distinct R groups are covalently linked to each other.

31. The film of claim 26, wherein the organic acid is selected from lactic, citric, and hexanoic acids.

32. The film of claim 26, wherein the organic acid is lactic acid.

33. The film of claim 12, further comprising a lubricant.

34. The film of claim 12, wherein the beneficial agent is an ionizable agent.

35. The film of claim 12, wherein the beneficial agent is a contraceptive agent.

36. The film of claim 35, wherein the contraceptive agent is a spermicidal agent.

37. The film of claim 36, wherein the spermicidal agent is selected from nonylphenoxypolyethoxy ethanol, p-diisobutylphenoxy polyethanol, benzalkonium chloride, p-methanyl phenylpolyoxyethylene ether, chlorhexidine, polyoxyethylene oxypropylene stearate, ricinoleic acid, glycerol ricinoleate, and methyl benzethonium chloride.

38. The film of claim 36, wherein the spermicidal agent is selected from nonylphenoxypolyethoxy ethanol, p-diisobutylphenoxy polyethanol, benzalkonium chloride, and p-methanyl phenylpolyoxyethylene ether.

39. The film of claim 36, further including a therapeutic agent.

40. The film of claim 39, wherein the therapeutic agent is selected from anti-inflammatory agents and anti-infective agents.

41. The film of claim 39, wherein the therapeutic agent is an anti-infective agent.

42. The film of claim 41, wherein the anti-infective agent is an anti-viral agent.

43. The film of claim 41, wherein the anti-infective agents is an anti-retroviral agent.

44. The film of claim 41, wherein the anti-infective agents is an anti-herpes agent.

45. The film of claim 41, wherein the anti-infective agent is an anti-bacterial agent.

46. The film of claim 41, wherein the anti-infective agent is an anti-fungal agent.

47. The film of claim 39, wherein the therapeutic agent is an anti-inflammatory agent.

48. The film of claim 12, further comprising a lubricant.

49. The film of claim 12, wherein the beneficial agent is a lubricant.

50. A pH-responsive composite film, comprising a laminated composite of (a) a bioadhesive layer that serves to affix the film to a mucosal surface within the vagina and, laminated thereto, (b) a reservoir layer comprising a beneficial agent and a biocompatible hydrophilic polymer.

51. The composite film of claim 50, wherein the reservoir layer comprises a first layer having the beneficial agent and a second layer having the biocompatible hydrophilic polymer.

52. The composite film of claim 51, wherein the first layer further comprises a controlled release polymer and the second layer further comprises a pH-responsive material.

53. The composite film of claim 51, wherein the biocompatible hydrophilic polymer is a pH-responsive material.

54. The composite film of claim 52, wherein the controlled release polymer is selected from carbomers, poly(alkylene oxides), and cellulose ethers.

55. The composite film of claim 54, wherein the poly(alkylene oxide) is poly(ethylene oxide) and the cellulose ether is hydroxypropyl methylcellulose.

56. The composite film of claim 51, wherein the biocompatible hydrophilic polymer is a controlled release polymer.

57. The composite film of claim 52, wherein the pH-responsive material is selected from cellulosic polymers; acrylic acid polymers and copolymers; vinyl polymers and copolymers; and shellac.

58. The composite film of claim 50, wherein the reservoir layer comprises a first region containing one beneficial agent and a second region containing a second beneficial agent.

59. The composite film of claim 58, comprising a bilayer in which the first and second regions are layers.

60. A method of treating or preventing pH-responsive diseases in a female individual, comprising:
(a) positioning in the vaginal passage of the female individual a pH-responsive film for the administration of a beneficial agent, wherein the pH-responsive film comprises an effective amount of an ionizable beneficial agent; and, a biocompatible, hydrophilic polymer that is positively charged at a first pH and in electronically neutral form at a higher pH; and
(b) administering an effective amount of the beneficial agent into the vaginal passage when the pH is equal to or above 7.

61. A method of contraception in a female individual, comprising:
(a) positioning in the vaginal passage of the female individual prior to sexual intercourse a pH-responsive film for the administration of a beneficial agent, wherein the pH-responsive film comprises an effective amount of an ionizable beneficial agent; and, a biocompatible, hydrophilic polymer that is positively charged at a first pH and in electronically neutral form at a higher pH; and
(b) administering an effective amount of the beneficial agent into the vaginal passage the pH is equal to or above 7.
